(19) United States
(12) Patent Application Publication
(10) Pub. No.: US 2025/0120857 A1
(43) Pub. Date: Apr. 17, 2025

(54) Her-ik

(71) Applicant: Natasha Anita Hall, Brooklyn, NY (US)

(72) Inventor: Natasha Anita Hall, Brooklyn, NY (US)

(73) Assignee: Herplum LLC, Brooklyn, NY (US)

(21) Appl. No.: 18/651,864

(22) Filed: May 1, 2024

Related U.S. Application Data
(63) Continuation of application No. 63/590,422, filed on Oct. 14, 2023.

(30) Foreign Application Priority Data
Oct. 14, 2023 (US) 63/590422

(51) Int. Cl. A61F 13/472

(52) U.S. Cl. CPC A61F 13/47218

(57) ABSTRACT

A menstrual and incontinence product that can be described as a pad, menstrual pad, incontinence pad, sanitary napkin, sanitary pad, menstrual underwear, underwear. The menstrual product encompasses a 3-D fin-like area that's located to fit between the perineum and the anus. The fin-like 3-D area will be wedged between the buttock to reduce back leakage during menstruation.

### DESCRIPTION OF DESIGN

A menstrual product that can be described as a pad, menstrual pad, sanitary napkin, sanitary pad, menstrual underwear, underwear. The menstrual product encompasses a 3-D fin-like area that's located to fit between the perineum and the anus. The fin-like 3-D area will be wedged between the buttock to reduce back leakage during menstruation.

An incontinence product that can be described as a pad, incontinence pad, sanitary napkin, sanitary pad, incontinence underwear, underwear. The incontinence product encompasses a 3-D fin-like area that's located to fit between the perineum and the anus. The fin-like 3-D area will be wedged between the buttock to reduce back leakage during incontinence.

### DETAILED DESCRIPTION OF DESIGN

Outlining Shape: Mirrors the curvatures of a woman's exterior body shape.
The top of the pad, similar to the head of a human, has the smallest width and provides alignment for the rest of the pad.
The wings of the pad, similar to the arms of a human, are used to wrap around the crotch of the person(s) underwear, hugging the underwear to ensure it remains in place and is comfortable.
Alongside and underneath the wings is similar to the torso/core, which provides stability in the center of the pad.
The pad then expands in width, similar to the hips, creating structure and provides buttock coverage during usage.
The pad then decreases in width, similar to the legs/feet like a tail to fit comfortably up the back and in between the buttocks.

### BRIEF DESCRIPTION OF THE DRAWINGS

It provides support FIG. 1) in between the perineum and the anus, wedged between the buttocks,

FIG. 2) support between the inner leg and the labia

FIG. 3) It folds within itself into the pocket and

FIG. 4) a hook at the bottom/tail end

FIG. 1 is the side view of the 3-D fin-like area that's located to fit between the perineum and the anus, to be wedged between the buttocks to reduce back leakage, which happens when vaginal fluid flows towards the rear;

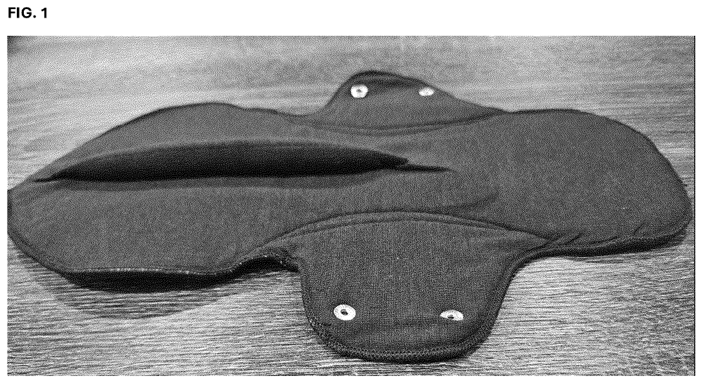

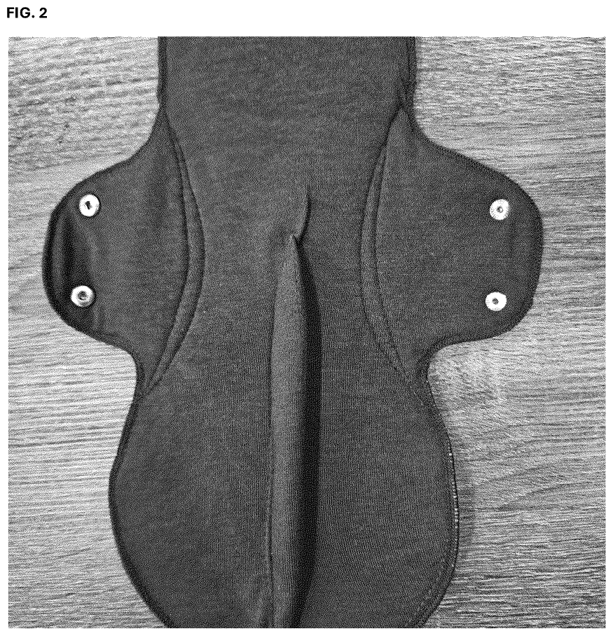

FIG. 2 is the top view of the elevated curved arches near the wings on each side to provide support in reducing side leakage, which happens when there's vaginal fluid that flows between the inner thigh and the labia due to gushes of vaginal discharge being secreted from the vagina therefore preventing vaginal discharge from leaking onto any garments;

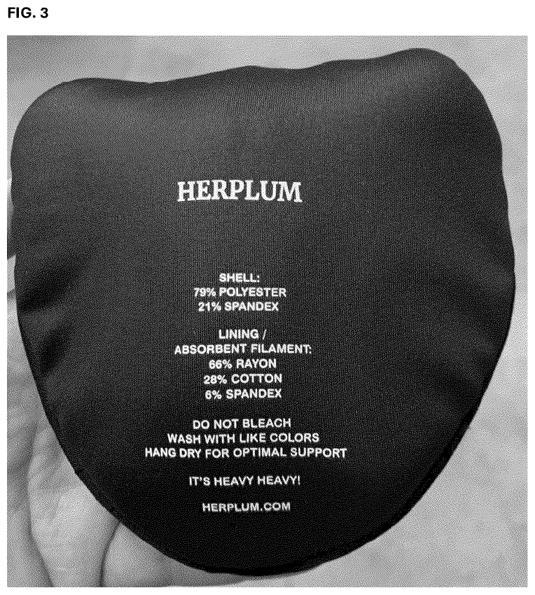

FIG. 3 is an encapsulated view of the menstrual or incontinence product folded within the pocket for storage and transport, which allows for the blood or urine to remain contained without leaking onto or into the carrying case, while also allowing for the pad or underwear to remain clean and organized while being stored; and

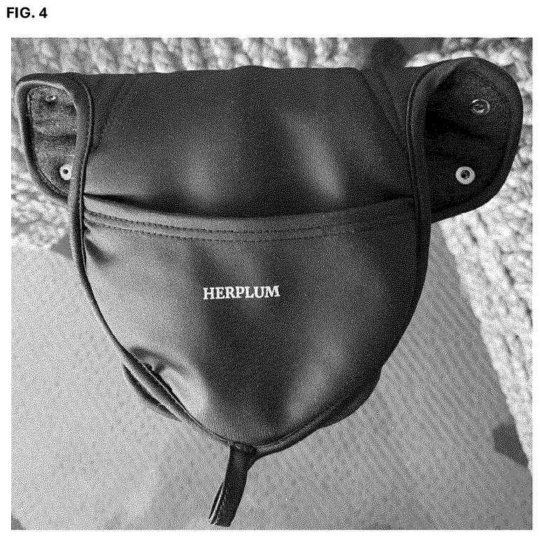

FIG. 4 is a folded pocket view capturing the hook at the bottom or tail-end of the pad or underwear which allows airflow to be evenly distributed during the drying process.

Absorbency/Fabric Layering: Utilizes natural absorbent fabrics to complement the natural liquid secretions of the body.
Top Layer, closest to the skin/vagina, is a bamboo/cotton fabric.
   Used for its softness and comfortably.
Middle Layers (Absorbency Layers), is in between the top layer and the outermost layer, is a bamboo/fleece fabric.
   Used to absorb the natural bodily secretions of blood, urine and/or vaginal discharge.
Outermost Layer, the furthest away from the skin/vagina, is a durable water repellent (DWR) plant based polyester fabric.
   Used to reduce the leakage onto the person(s) garments.

### Frequently Asked Questions (FAQ's)

Why does this product function better than other products on the market?
This product specifically solves for back leakage and side leakage during menstruation or incontinence.
   Back leakage happens when either blood, urine, and/or other bodily secretions:
      a) flows between the buttocks onto the lower back
      b) flows between the buttocks onto the upper butt.
      c) flows between the buttocks onto the person(s) garments.
      d) flows between the buttocks on the person(s) bedding or furniture and/or
      e) flows between the buttocks onto any material or organ that is not a part of the pad.
   Side leakage happens when either blood, urine, or other bodily secretions:
      a) flows between the inner thigh and the labia onto the inner leg.
      b) flows between the inner thigh and the labia up to the Mons Pubis.
      c) flows between the inner thigh and the labia backwards towards the lower buttocks and/or
      d) flows between the inner thigh and the labia onto any material or organ that is not a part of the pad

How does this product work?
The pad (ex. Sanitary pad, Sanitary napkin, menstrual pad, incontinence pad) is an instrument that is worn, attached around a person(s) underwear, during menstruation or incontinence to absorb blood, urine, or other bodily secretions. The pad is attached around the underwear using snaps, Velcro, or other trim for fastening the wings together. The person(s) wearing the pad will place the 3-D fin-like area between the buttocks, which will fit between the perineum and the anus to reduce back leakage. The elevated side arches will provide support in reducing side leakage. Post usage of the pad, it can be encapsulated within the pocket for transport or storage.

How are the underwear's different and/or similar to the pad?
The similarities between the menstrual or incontinence pad (ex. Sanitary pad, Sanitary napkin, menstrual pad, incontinence pad) and the menstrual or incontinence underwear (ex. menstrual underwear, incontinence underwear) are:
   There is a 3-D fin-like area that's located to fit between the perineum and the anus. The fin-like 3-D area will be wedged between the buttock to reduce back leakage during usage.
   There are elevated curved arches on each side to provide support in reducing side leakage. When there are gushes of blood, urine, and/or vaginal discharge being secreted from the vagina the elevated curved arches provide additional absorbency to prevent the bodily secretions from leaking onto the person(s) garments.
The differences between the menstrual or incontinence pad and the menstrual or incontinence underwear are:
   The underwear is one piece that is worn to cover the hips, vagina, and buttocks.
   The pad (ex. Sanitary pad, Sanitary napkin, menstrual pad, incontinence pad) is attached to the crotch of the underwear of the person(s) whereas the underwater (ex. menstrual underwear, incontinence underwear) has the features of the pad built into the underwear.
      The features built into the underwear include:
         There is a 3-D fin-like area that's located to fit between the perineum and the anus. The fin-like 3-D area will be wedged between the buttock to reduce back leakage during usage.
         There are elevated curved arches on each side to provide support in reducing side leakage. When there are gushes of blood or urine being secreted from the vagina the elevated curved arches provide additional absorbency to prevent the blood and/or urine to leak on the person(s) garments.

How long can one pad or underwear be used?
Depending on the person(s) blood, urine flow, or other bodily secretions the pad or underwear can be used for up to 10 to 12 hours.

The invention description above is a substitute specification that does not contain any new matter that was described with the original filing date May 1, 2024 for application Ser. No. 18/651,864, 65/390,422 and 29/940,312.

The application was made or was authorized to be made by Natasha Hall and Natasha Hall believes herself to be the original inventor of a claimed invention in the application. The above detailed description of the invention, Her-Ik, is the invention.

1. A menstrual or incontinence product that can be characterized as a pad, menstrual pad, incontinence pad, sanitary napkin, sanitary pad, menstrual underwear, incontinence underwear or underwear comprising of:
a 3-D fin-like area that's located to fit between the perineum and the anus, to be wedged between the buttocks to reduce back leakage, which happens when vaginal fluid flows towards the rear;
elevated curved arches near the wings on each side to provide support in reducing side leakage, which happens when there's vaginal fluid that flows between the inner thigh and the labia due to gushes of vaginal discharge being secreted from the vagina therefore preventing vaginal discharge from leaking onto any garments;
a pocket to encapsulate or fold the pad or underwear for storage and transport, which allows for the blood or urine to remain contained without leaking onto or into the carrying case, while also allowing for the pad or underwear to remain clean and organized while being stored; and
a hook at the bottom or tail-end of the pad or underwear which allows airflow to be evenly distributed during the drying process.
